(19) United States
(12) Patent Application Publication
(10) Pub. No.: US 2026/0174584 A1
(43) Pub. Date: Jun. 25, 2026

(54) SEQUENTIAL PENILE ELONGATION AND ANGULAR REALIGNMENT SYSTEM

(71) Applicant: The Government of the United States, as Represented by The Secretary of the Army, Fort Detrick, MD (US)

(72) Inventors: Ronald Caras, Gig Harbor, WA (US); Carolyn Salter, Dupont, WA (US); Timothy Wright, Tacoma, WA (US); Theodore Crisostomo-Wynne, Lakewood, WA (US)

(21) Appl. No.: 18/862,260

(22) PCT Filed: Apr. 26, 2023
(86) PCT No.: PCT/US2023/019963

§ 371 Date: Nov. 1, 2024

Related U.S. Application Data
(60) Provisional application No. 63/338,432, filed on May 4, 2022.

(51) Int. Cl. A61F 5/41

(52) U.S. Cl. CPC A61F 5/41; A61F 2005/411; A61F 2005/412

(57) ABSTRACT

Methods and apparatus for treating Peyronie's disease. In one embodiment, the method includes providing a plurality of inserts (34) with each insert (34) having a cavity (36) formed therein. Each cavity (36) is formed to receive and encapsulate the entire angulated penis of the patient. The degrees of angulation of the respective cavities (36) of the plurality of inserts (34) are a series of sequentially decreasing degrees of angulation that are all less than the initial degree of angulation of the angulated penis. The plurality of inserts (34) is used by the patient sequentially to gradually break down fibrotic plaque and gradually reshape the angulated penis with minimal discomfort to the patient. A stiff cylindrical housing (32) receives the plurality of inserts (34) in a sequential manner and creates a vacuum chamber (46) above each insert.

### STATEMENT OF GOVERNMENT INTEREST

The invention described herein may be manufactured, used and licensed by or for the United States Government.

### BACKGROUND OF THE INVENTION

The invention relates to apparatus and methods for treating Peyronie's disease.

FIG. 1A is a cutaway view of a normal erect human penis 10 showing the corpora cavernosa 12 and the corpus spongiosum 14. The corpora cavernosa 12 are two parallel tubes or columns of spongy tissue. Increased blood flow to the relatively elastic corpora cavernosa 12 and the corpus spongiosum 14 cause an erect penis. FIG. 1B is a cutaway view of an erect human penis 16 with the arrow showing the penile curvature caused by Peyronie's disease. FIG. 1C is a sectional view taken along the line 1C-1C of FIG. 1B. As seen in FIGS. 1B and 1C, Peyronie's disease is a medical condition in which angulation of the penis 16 is caused by fibrotic plaque formation 18 of the corpora cavernosa 12. The fibrotic plaque 18 causes the penis 16 to bend during erection. The bent erection can cause patient or partner pain or limit the ability of a male to have sexual intercourse. If an area of the corporal bodies 12 does not expand due to scarring, the penis 16 will cantilever over on this point of fibrosis thereby causing deformity and lack of function. The prevalence of Peyronie's varies in the literature from 2-9%, likely due to varying methods of diagnosis, from self-reported to clinical diagnosis. See, for example, Chung E., Ralph D., Kagioglu A. et al., Evidence-Based Management Guidelines on Peyronie's Disease, J Sex Med. 2016; 13: 905-23.

Peyronie's disease has limited treatment options. These options include intralesional injections, invasive surgeries and traction devices. These treatment options have undesirable side effects and marginal success rates. Risks of invasive surgery include decreased penis length, sensation loss and erectile dysfunction. Conventional traction devices have limited evidence of success. Most conventional traction devices are thought to prevent worsening of curvature and there is limited data on improvement of Peyronie's disease. A need exists for a more effective and safe treatment for Peyronie's disease.

### SUMMARY OF THE INVENTION

An apparatus for treating a patient having Peyronie's disease may include a housing that is hollow, and a first insert removably disposed in the housing. The first insert has a cavity for insertion of an angulated penis of the patient wherein a degree of angulation of the cavity is less than an initial degree of angulation of the angulated erect penis of the patient. A first fluid conduit in the first insert has one end that connects with the cavity wherein the first fluid conduit extends to an exterior of the first insert. The housing includes a first end that is closed to define a vacuum chamber in the housing between the first insert and the first end of the housing. A second fluid conduit has one end that extends through the first end of the housing to the vacuum chamber and a second end that is external to the housing. A release valve may be located in the second fluid conduit external to the housing for selectively releasing a vacuum in the vacuum chamber. A diaphragm has an opening therein for insertion of the angulated penis in the cavity. The diaphragm is located at a second end of the housing corresponding to a base of the angulated erect penis to thereby provide a vacuum seal for the cavity.

The cavity may completely encapsulate the angulated erect penis from the base of the angulated erect penis to the tip of the angulated erect penis. The cavity has a shape such that an interior of the cavity contacts and applies pressure to the angulated erect penis at least at an area of undesired angulation to thereby gradually break down fibrotic plaque and gradually reshape the angulated erect penis.

The apparatus may further include a vacuum pump removably attached to the second end of the second fluid conduit for creating the vacuum in the vacuum chamber and the cavity. When the angulated penis is inserted through the diaphragm and into the cavity, the vacuum pump creates a vacuum causing erection of the angulated penis in the cavity, a gradual break down of the fibrotic plaque in the angulated erect penis and a gradual reshaping of the angulated erect penis so that a degree of angulation of the angulated erect penis outside of the cavity gradually becomes a same degree as the degree of angulation of the cavity.

The apparatus may further comprise a plurality of removable inserts for sequential disposal in the housing. Each of the plurality of removable inserts includes a cavity for receiving the angulated penis. The degrees of angulation of the respective cavities of the plurality of removable inserts are a sequence of decreasing degrees of angulation to thereby gradually straighten the angulated erect penis of the patient. The shapes of the respective cavities are such that the interiors of the respective cavities contact and apply pressure to the angulated erect penis at least at an area of undesired angulation.

The degree of angulation in the cavity of the first insert may be in a range of about 5% to about 15% less than the initial degree of angulation of the angulated erect penis of the patient.

In a method of treating a patient having Peyronie's disease, the patient has an angulated erect penis with a first degree of angulation. The method includes providing a plurality of inserts wherein each insert has a cavity formed therein. Each cavity is formed to receive and encapsulate the entirety of the angulated erect penis of the patient from base to tip. Degrees of angulation of the respective cavities of the plurality of inserts are a series of sequentially decreasing degrees of angulation that are all less than the first degree of angulation of the angulated erect penis to thereby gradually break down fibrotic plaque and straighten the angulated erect penis.

The method includes inserting the angulated penis of the patient into the cavity of a first one of the plurality of inserts having the largest degree of angulation that is smaller than the first degree of angulation of the angulated erect penis. A vacuum is created in the cavity of the first one of the inserts to cause the angulated penis to become erect such that the cavity encapsulates the entirety of the angulated erect penis from the base to the tip. The interior of the cavity contacts and applies pressure to the angulated erect penis at least at an area of undesired angulation of the angulated erect penis. The vacuum is maintained in the cavity of the first one of the inserts with the angulated erect penis inserted therein for a period of time to gradually break down fibrotic plaque at the area of undesired angulation and decrease the first degree of angulation of the angulated erect penis. Then, the vacuum in the cavity is released and the angulated penis is removed from the cavity.

In one embodiment, the method includes placing the first one of the inserts in an external cylindrical housing that includes a vacuum chamber connected to the cavity of the first one of the inserts.

The method further includes repeating the steps of inserting, creating a vacuum, maintaining the vacuum, releasing the vacuum and removing the angulated penis from the cavity, until a degree of angulation of the angulated erect penis is the same degree as the degree of angulation of the cavity of the first one of the plurality of inserts.

The method may further comprise inserting the angulated penis into the cavity of a second one of the plurality of inserts having a next largest degree of angulation that is smaller than the degree of angulation of the cavity of the first one of the plurality of inserts. A vacuum is created in the cavity of the second one of the inserts to cause the angulated penis to become erect such that an interior of the cavity contacts and applies pressure to the angulated erect penis at least at the area of undesired angulation. The vacuum is maintained in the cavity of the second one of the inserts with the angulated erect penis inserted therein for a period of time to gradually decrease the degree of angulation of the angulated erect penis. The vacuum in the cavity is released and the angulated penis is removed from the cavity. Then, the method includes repeating the steps of inserting, creating a vacuum, maintaining the vacuum, releasing the vacuum and removing the angulated penis from the cavity until a degree of angulation of the angulated erect penis is the same degree as the degree of angulation of the cavity of the second one of the plurality of inserts.

The method may further comprise sequentially using an insert of the plurality of inserts having a cavity with a next smallest degree of angulation than a cavity of the previously used insert, until the angulated erect penis has obtained a desired angulation.

A kit for treatment of Peyronie's disease may include a series of inserts having respective cavities. The respective cavities of the inserts are configured for insertion of an angulated penis and for encapsulation of an angulated erect penis of a patient from a base to a tip of the angulated erect penis. The degree of angulation of the respective cavities of sequential inserts decreases. The kit may include a hollow, cylindrical housing for serially receiving the series of inserts starting with an insert having a cavity with a largest degree of angulation and ending with an insert having a cavity with a smallest degree of angulation. The housing includes a first end that is closed to define a vacuum chamber in the housing between each one of the series of inserts and the first end of the housing. Each insert may have a first fluid conduit that extends from the cavity to an exterior of the insert. A second fluid conduit may have one end that extends through the first end of the housing to the vacuum chamber and a second end that is external to the housing. A release valve may be located in the second fluid conduit external to the housing for selectively releasing a vacuum in the vacuum chamber. A diaphragm in the kit may have an opening therein for insertion of an angulated penis in the cavity of each insert. The kit may include a vacuum pump.

In one embodiment, the kit may include a holding bracket to use for assessing the curvature or angulation of a penis. The holding bracket may include an inner annular member that is configured to extend circumferentially around the hollow, cylindrical housing and an outer annular member rotatably fixed to the inner annular member such that the outer annular member rotates with respect to the inner annular member. The holding bracket may include an arm that is fixed to and extends radially away from the outer annular member. A clamp may be fixed to the arm and configured to hold a device that includes a camera. The clamp may be adjustable to hold varying widths of devices.

In another aspect of the invention, an apparatus for treating a patient having Peyronie's disease includes a pliable vessel having a central longitudinal axis. The pliable vessel may comprise a tubular portion made of a spiral coil embedded in an interior layer of a first material and an exterior layer of a second material and capable of maintaining a vacuum therein. The tubular portion may taper at the top end to close the tubular portion. An interior of the tubular portion is configured to encapsulate an entirety of an angulated erect penis of the patient from a base of the angulated erect penis to a tip of the angulated erect penis. A bottom closure seals the bottom of the tubular portion and a diaphragm is disposed in the bottom closure for insertion of a penis of the patient into the interior of the tubular portion. A vacuum pump connection may be connected to the interior of the tubular portion.

The apparatus includes at least one bending assembly for changing an angle of the central longitudinal axis of the pliable vessel and thereby bending the pliable vessel such that an interior surface of the tubular portion contacts and applies pressure to the angulated erect penis of the patient at least at an area of undesired angulation to thereby gradually break down fibrotic plaque and gradually reshape the angulated erect penis.

The at least one bending assembly may include first and second rods that intersect at an angle adjustment assembly. The at least one bending assembly may include at least one rod-holding ring for each of the first and second rods. The at least one rod-holding ring encircles the tubular portion, is fixed to the tubular portion and includes a swiveling snap for retaining one of the rods to the at least one rod-holding ring.

The angle adjustment assembly may include a pair of disks. Each of the pair of disks has teeth circumferentially located on surfaces of the disks that face each other wherein the teeth on one disk engage the teeth on the other disk. A sleeve may be fixed on each of non-facing surfaces of the disks wherein each sleeve receives one of the first and second rods and allows translation of the rod through the sleeve. A fastener removably fixes the pair of disks together.

A vacuum pump may be removably attached to the vacuum pump connection for creating the vacuum in the tubular portion. When the angulated penis is inserted through the diaphragm and into the tubular portion, the vacuum pump creates a vacuum causing erection of the angulated penis in the tubular portion and a gradual break down of the fibrotic plaque in the angulated erect penis and a gradual reshaping of the angulated erect penis so that a degree of angulation of the angulated erect penis outside of the tubular portion gradually becomes the same degree as a degree of angulation of the pliable vessel.

### BRIEF DESCRIPTION OF THE DRAWINGS

In the drawings, which are not necessarily to scale, like or corresponding parts are denoted by like or corresponding reference numerals.

FIG. 1A is a cutaway view of a normal erect human penis.

FIG. 1B is a cutaway view of an erect human penis showing the penile curvature caused by Peyronie's disease.

FIG. 1C is a sectional view taken along the line 1C-1C of FIG. 1B.

FIG. 2 is a schematic illustration showing a plurality of inserts and a time sequence of straightening of an angulated erect penis.

FIG. 3 is an exploded perspective view of an embodiment of an apparatus for straightening an angulated penis.

FIG. 4 is a longitudinal cutaway view of FIG. 3.

FIG. 5A is a bottom view of a diaphragm.

FIG. 5B is a perspective view of FIG. 5A.

FIG. 6 illustrates an embodiment of a hand-operated vacuum pump.

FIG. 7 illustrates an embodiment of an electrically powered vacuum pump.

FIG. 8A illustrates an embodiment of an apparatus for assessing the curvature of a penis.

FIG. 8B is an enlarged sectional view of the annular members of the apparatus of FIG. 8A

FIG. 9A illustrates another embodiment of an apparatus for straightening an angulated penis.

FIG. 9B is an exploded view an angle adjustment assembly.

FIG. 9C is a partial view of a rod-holding ring with a swiveling snap connector.

FIG. 9D is a cross-sectional view of a tubular portion of the apparatus of FIG. 9A.

### DETAILED DESCRIPTION

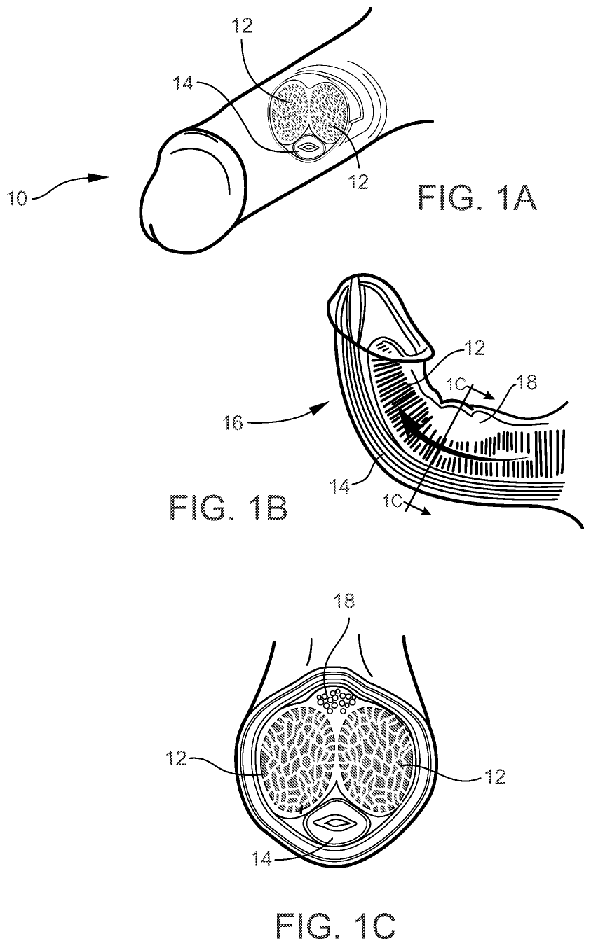

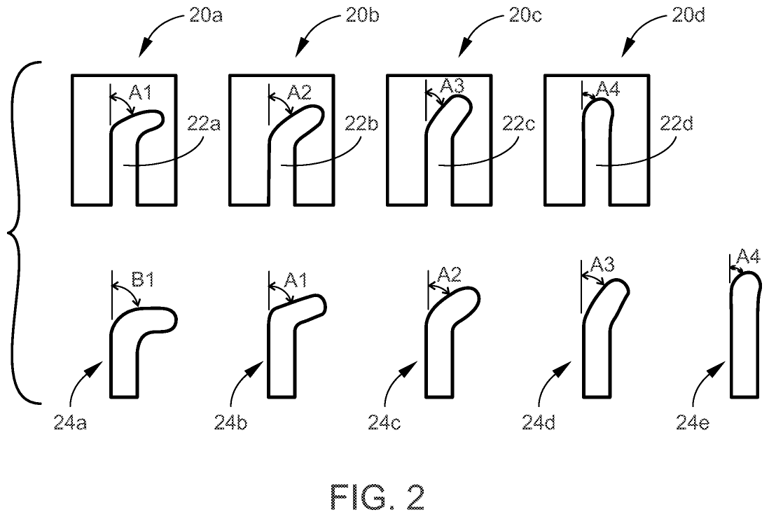

The invention includes apparatus and methods for treating Peyronie's disease. The plasticity of the fibrotic plaque 18 (FIGS. 1B and 1C) in the penis 16 of a patient with Peyronie's disease enables sequential modelling of the penis from a bent state to a more normal, straight conformation. A vacuum erection device, which may be individually-tailored, is used to sequentially model the penis by swapping inserts in the device to decrease the angulation of the penis over time. Each insert of the device includes a cavity for the erect penis and is able to model the erect penis in several axes simultaneously when the penis is erect, rather than flaccid. Modelling of the erect penis is more effective than modelling the flaccid penis. However, hand modeling the erect penis can lead to injury, so the gentle, gradual modeling of the erect penis in accordance with the invention may maximize benefit while limiting risks. The cavity in each of the plurality of inserts may be custom tailored to fit the penis of each individual patient.

Referring to FIG. 2, a general description of the operation of an embodiment of the invention involves a plurality of inserts 20a, 20b, 20c, 20d each having a respective cavity 22a, 22b, 22c, 22d for insertion of a penis. Cavities 22a, 22b, 22c, 22d each have a respective angle A1, A2, A3, A4 that decreases sequentially from A1 to A2 to A3 to A4. Angle A4 may be a desired minimum angle. In general, a functionally straight penis is defined as having less than about 20 degrees angulation. So, angle A4 (or the angle of the last insert if more or less than four inserts are used) is desired to be at least less than 20 degrees. Below each insert 20a, 20b, 20c, 20d is shown a time series of sequentially straightening an angulated erect penis through states 24a, 24b, 24c, 24d and 24e. In state 24a, the angulated erect penis has not been treated and has the greatest angulation B1.

The penis in state 24a is first inserted in cavity 22a of insert 20a, which has an angle A1 that is less than angle B1. A vacuum is created in cavity 22a to put the penis in state 24a in an erect state and the vacuum is then held for a length of time, for example, about 30 minutes once a day. As discussed further below, periods of times other than 30 minutes may be used and the device may be used more or less than once daily, depending on various factors. This procedure may be repeated, for example, daily, until the angle B1 of the penis in state 24a has decreased to angle A1 of cavity 22a. Then, the penis is in state 24b and has angulation A1. The process is repeated using insert 20b and cavity 22b. That is, the penis in state 24b is inserted in cavity 22b having an angle A2 that is less than angle A1. A vacuum is created in cavity 22b to put the penis in state 24b in an erect state and the vacuum is held for a length of time, for example, 30 minutes. This procedure is followed, for example, daily, until the angulation of the penis in state 24b becomes angle A2, as shown by state 24c. The procedure is again followed for insert 20c until the penis in state 24c has angle A3, as shown by state 24d. The procedure is followed again with insert 20d until the penis reaches state 24e and has an angulation of A4. Angle A4 may be a desired minimum angle of at least less than about 20 degrees, as discussed above. The number of inserts and angles shown are exemplary only and fewer or more steps of the procedure may be appropriate for a specific patient.

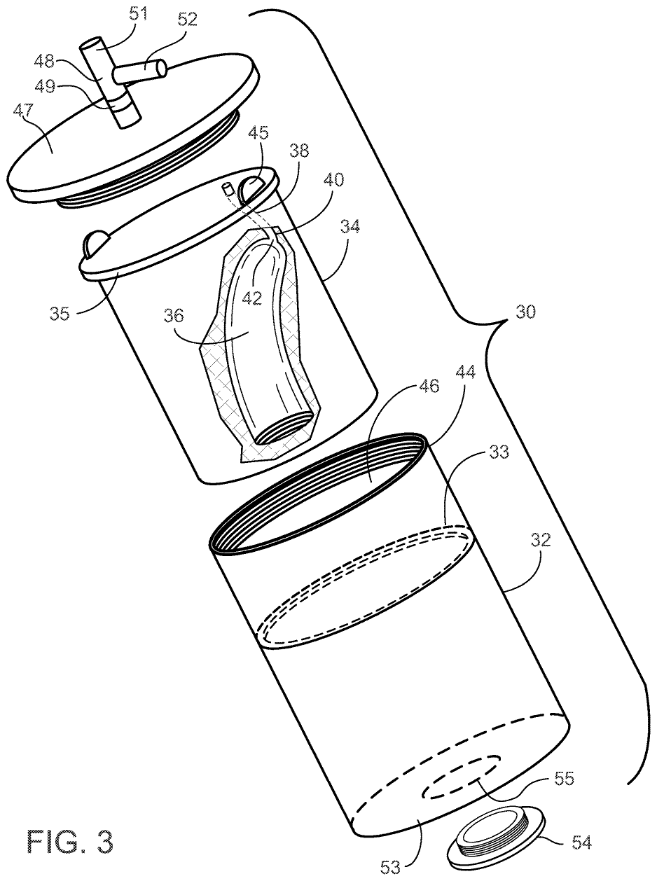

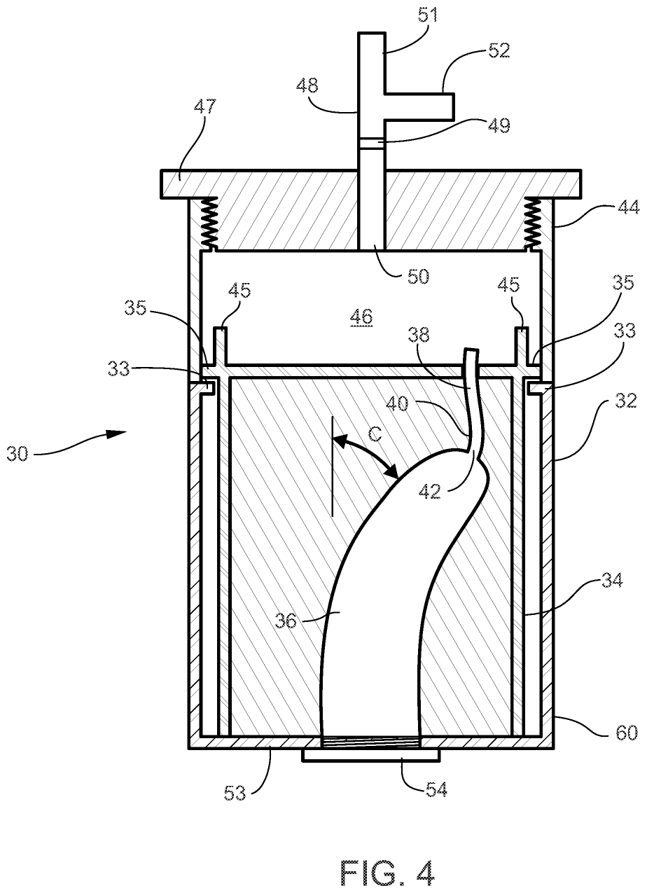

FIG. 3 is an exploded perspective view of an embodiment of an apparatus 30 for treating a patient having Peyronie's disease. FIG. 4 is a longitudinal cutaway view of FIG. 3. Apparatus 30 includes a hollow housing 32 and an insert 34 removably disposed in and nesting in the housing 32. Housing 32 may be made of a hard or stiff plastic material. Housing 32 may be transparent. Housing 32 and insert 34 may have generally cylindrical shapes. Housing 32 may include a ledge 33 that extends around an inner circumference of the housing. Insert 34 may include a circumferential flange 35 that rests upon ledge 33 when insert 34 is disposed in housing 32. The insert 34 defines a cavity 36 for insertion of an angulated penis of a patient. A degree C. of angulation in the cavity 36 (FIG. 4) is less than a degree of angulation of the untreated angulated erect penis of the patient.

Cavity 36 is shaped so that the interior of the cavity 36 contacts and applies pressure to at least the area of the angulated erect penis that has the undesired angulation. In the case of Peyronie's disease, the area of undesired angulation is where the fibrotic plaque 18 (FIGS. 1B and 1C) has formed. Thus, when the angulated penis is erect and in cavity 36, the interior of the cavity bears upon the area of the penis at and surrounding the fibrotic plaque 18 to gradually break down the fibrotic plaque and reshape the penis. The cavity 36 may contact additional areas of the angulated erect penis. The cavity 36 completely encapsulates the angulated erect penis from its base at the pelvic area to its tip.

The removable insert 34 includes a first fluid conduit 38. First fluid conduit 38 has one end 40 that connects with the cavity 36. In one embodiment, the one end 40 of the first fluid conduit 38 connects with the cavity 36 at a point 42 corresponding to a tip of the angulated erect penis. First fluid conduit 38 extends through removable insert 34 to the exterior of the insert.

The housing 32 includes a first end 44 that is closed to define a vacuum chamber 46 in the housing between the removable insert 34 and the first end of the housing. To close the first end 44 of the housing 32, a top or lid 47 may be removably fixed to first end 44 by a threaded connection, for example. That is, the first end 44 of housing 32 may have internal threads and the lid 47 may have external threads that engage the internal threads. A second fluid conduit 48 has one end 50 that extends through lid 44 of housing 32 to the vacuum chamber 46 and a second end 51 that is external to the housing. A release valve 52 is located in the second fluid conduit 48 external to the housing 32 for selectively releasing a vacuum in the vacuum chamber 46. Release valve 52 is especially important if the patient experiences pain from an adverse effect of the vacuum in cavity 36. Release valve 52 may, for example, be rotated around the axis of conduit 48 to selectively open and close conduit 48 to ambient air. A fluid lock-out valve 49 may be disposed internally in the second fluid conduit 48 nearer to the vacuum chamber 46 than the release valve 52. The fluid lock-out valve 49 is for cleanliness and prevention of urine leak into the release valve 52 for patients that have incontinence.

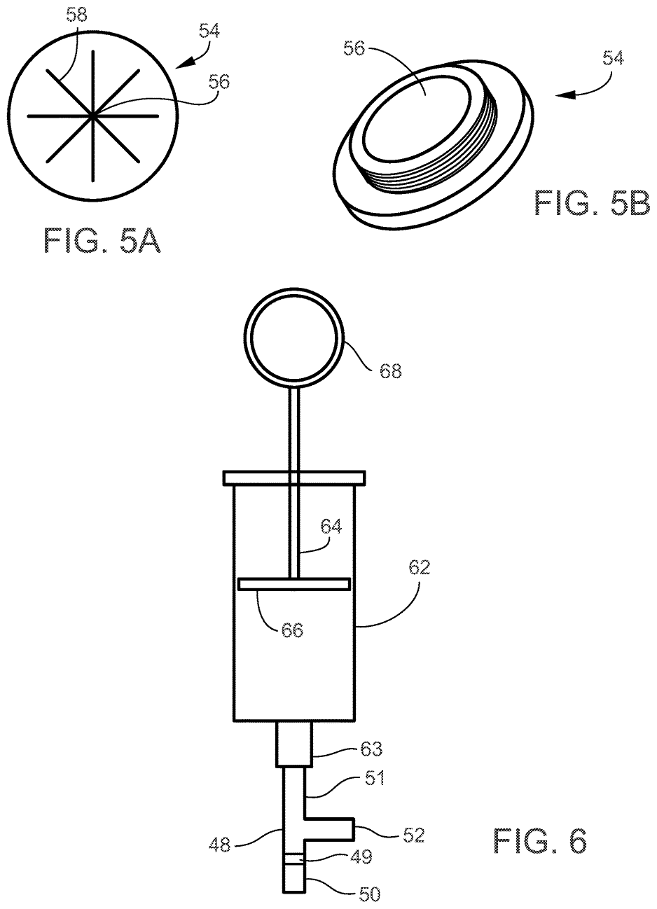

The second end 60 of housing 32 includes a bottom 53 that closes the second end of the housing. Bottom 53 includes an opening 55. A diaphragm 54 is placed in opening 55. Diaphragm 54 includes an opening 56 therein for insertion of the angulated penis in cavity 36. FIG. 5A is a bottom view of one embodiment of diaphragm 54 (as oriented in FIG. 4) and FIG. 5B is a perspective view of FIG. 5A. Opening 56 may be defined by a plurality of intersecting slits 58. The diaphragm 54 at the bottom 53 of housing 32 corresponds to the location of the base of the angulated erect penis when the penis is inserted in cavity 36, to thereby provide a vacuum seal for the cavity. Diaphragm 54 may be made of, for example, a rubber-like material or another elastomer.

A vacuum pump is removably attached to the second end 51 of the second fluid conduit 48 for creating the vacuum in the vacuum chamber 46 and the cavity 36. FIG. 6 shows a vacuum pump in the form of a hand-operated syringe 62. Syringe 62 may be connected to the second fluid conduit 48 by a Leur lock arrangement located at end 51 of conduit 48 and portion 63 of syringe 26. Syringe 62 may include a rod 64 connected to a reciprocating piston 66. A handle 68 is configured to allow the patient to grasp the handle 68 to reciprocate the piston 66 and create a vacuum in chamber 46 and cavity 36. Other types of manually-operated pumps may be used.

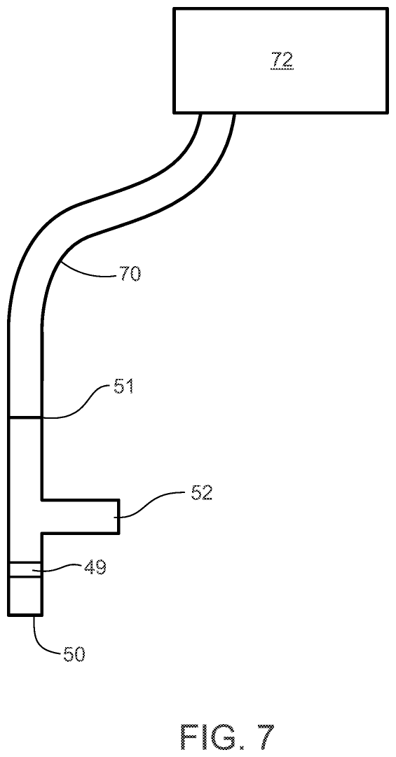

Alternatively, FIG. 7 shows an electrically-powered vacuum pump 72 connected to end 51 via tubing 71. Pump 72 may be powered by, for example, batteries that are rechargeable or not rechargeable or by a power supply connected to an alternating current outlet.

When the angulated penis is inserted through the diaphragm 54 and into the cavity 36, the vacuum pump is operated to create a vacuum. The vacuum causes erection of the angulated penis in the cavity 36. As the vacuum is maintained, the interior of the cavity 36 contacts and applies pressure to the erect penis at least at the area of undesired angulation to gradually break down fibrotic plaque at the area of undesired angulation and decrease the degree of angulation of the penis. Thus, a gradual reshaping of the angulated erect penis occurs. The gradual reshaping of the angulated erect penis results in the degree of angulation of the angulated erect penis outside of the cavity 36 gradually becoming the same degree as the degree of angulation of the cavity 36. The vacuum in vacuum chamber 46 should not exceed 200 mm Hg to avoid damage to the penis.

As described above with reference to FIG. 2, apparatus 30 includes a plurality of other removable inserts that fit in housing 32 in the manner of insert 34 and are sequentially disposed in the housing. Each of the plurality of removable inserts includes a cavity for receiving the angulated penis. The degrees of angulation of the respective cavities of the plurality of removable inserts are a series of decreasing degrees of angulation, to thereby gradually straighten the angulated erect penis of the patient. The number of inserts, the amount of penis angulation decreased by each insert and the amount of time required to straighten the penis at each step may vary widely depending on many factors including, for example, the initial angulation of the penis and the amount and strength of fibrotic plaque in the penis.

A proposed course of a treatment may include using the straightening device 30 for about thirty minutes per day every day. The device 30 may be applied by the patient without assistance from others. The appropriate insert, such as insert 34, is placed in the housing 32 so that the flange 35 of the insert 34 rests on the ledge 33 of the housing and the lid 47 is attached to the top of the housing. The penis is inserted in the cavity 36 of the insert 34 and the device 30 is fixed to the patient with, for example, a strap (not shown) that extends around the body of the patient. A vacuum pump, such as, for example, the hand-operated syringe 62 or electric pump 72, creates a negative pressure in the cavity 36 to make the penis erect within the device 30. As the penile deformity improves with repeated use, the patient will require additional inserts that reflect the decrease in penile angularity. Eventually, the angularity will reach a desired minimum angle (for example, less than about 20 degrees angulation). The duration of use of each insert 34 will vary depending on the needs of each patient. For example, inserts could be changed every 30 days or so, depending on the change in angulation. Total treatment time may be, for example, in the six month range, but it may be shorter or longer based on the initial degree of angulation and other patient factors.

One method of making the inserts involves custom fitting the inserts to a particular patient. This method enables modelling the penis along multiple planes simultaneously, if needed. The patient's erect penis is measured and then a series of custom-made inserts are made, for example, using a 3-D printer or mold. For ease of printing, the inserts may be made in two halves along a vertical plane (as oriented in FIG. 4). The material of the inserts may be, for example, a rubberized silicon or other suitable materials. For single planes of correction, inserts may be prefabricated for most common penis geometries. Three-dimensional modeling of the penis may be accomplished via mechanical techniques such as, for example, creating a silicone model. Other methods are possible, such as electronic scanning techniques. In addition to custom-made 3-D printed and molded inserts, and prefabricated inserts, other alternatives for inserts include stackable pre-printed circular insert portions that can be selected and stacked based on the patient's model. Inflatable inserts are another option.

Each insert may have an angle of curvature that is about 5% to about 15% less than the angle of curvature of the previously used insert. In one embodiment, each insert may have an angle of curvature that is 10% less than the angle of curvature of the previously used insert. For example, if the patient begins treatment with a sixty-degree dorsal curvature of the penis, the initial insert may have a cavity with an angulation that reflects about a 10% improvement over the baseline of sixty degrees and thus the cavity in the initial insert would have a fifty-four-degree curvature. The second insert would have a cavity with about a forty-nine-degree curvature. The third insert would have a cavity with about a forty-five-degree curvature and so forth until the curvature of the penis reaches a desired minimum curvature. The purpose of the sequential change is to prevent pain or injury to the penis that would occur by forcing the penis to be straight from an initial severe curvature. Further study is needed to optimize the frequency with which the sequential inserts are changed. One possibility is to change to a new insert monthly. The device 30 may also be used in conjunction with other therapies such as intralesional injections.

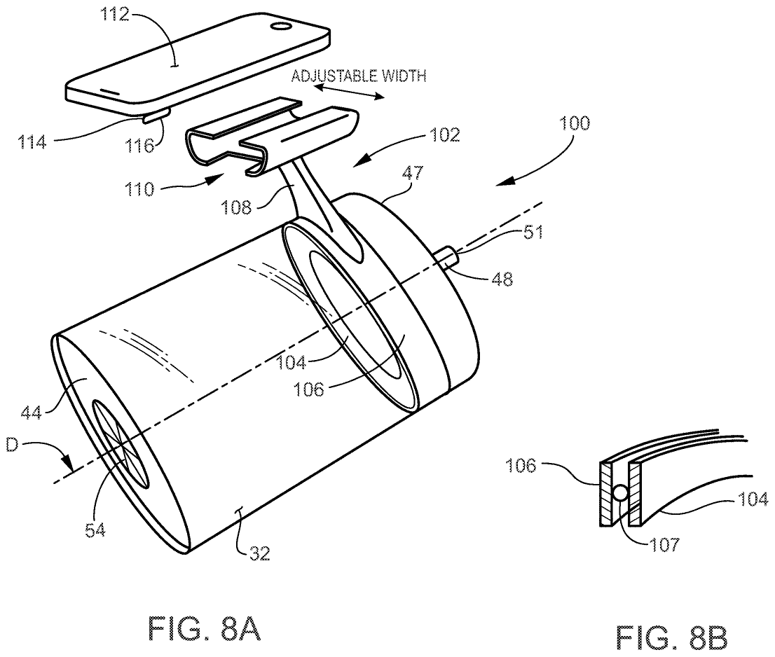

FIG. 8A illustrates an embodiment of an apparatus 100 for assessing the curvature of the penis of a patient. FIG. 8B is an enlarged sectional view of the annular members of the apparatus 100 of FIG. 8A. The apparatus 100 may include the hollow, cylindrical housing 32 as described with respect to FIGS. 3 and 4. The housing 32 is made of a transparent material, such as a stiff plastic. The diaphragm 54 is disposed in the opening 55 in the bottom of the first end 44 of the housing 32 and is configured for insertion of the penis in the housing. A vacuum pump attachment, such as the end 51 of the second fluid conduit 48 is disposed on the lid 47 of the housing 32. The second fluid conduit 48 may include the release valve 52 and the fluid lock-out valve 49 shown in FIGS. 3 and 4. For assessing the curvature of the penis of a patient, the housing 32 does not include an insert 34. Referring again to FIGS. 8A and 8B, a holding bracket 102 includes an inner annular member 104 removably fixed to the hollow, cylindrical housing 32 and extending circumferentially around the hollow, cylindrical housing. An outer annular member 106 is rotatably fixed to the inner annular member 104 such that the outer annular member rotates with respect to the inner annular member circumferentially around the hollow, cylindrical housing 32. Ball bearings 107 may be disposed between the inner and outer annular members 104, 106.

An arm 108 is fixed to and extends radially away from the outer annular member 106. A clamp 110 is fixed to the arm 108 and configured to hold a device 112 that includes a camera 114. Device 112 may be, for example, a cellular phone having a camera 114. The clamp 110 is adjustable to hold varying widths of devices 112. When device 112 is mounted in clamp 110, a camera lens 116 of the camera 114 faces radially inward toward a central longitudinal axis D of the housing 32 to thereby capture images of a penis inserted in the housing 32. Prior to capturing images of the penis in the housing, the vacuum pump is activated so that the penis is erect in the housing 32. The device 112 with camera 114 may be rotated around the housing 32 with the outer annular member 106 to capture images and/or video of the patient's penis. The images and/or video captured by camera 114 may be used by the patient and/or the patient's doctor to assess the degree of angularity of the patient's penis.

One aspect of the invention is a kit including the housing 32, lid 47, diaphragm 54, fluid conduit 48 with valves 49 and 52, a vacuum pump, and a plurality of inserts 34 that fit in the housing. Each insert 34 has a respective cavity for receiving the erect penis of the patient. Each cavity includes the fluid conduit 40. The respective cavities of the inserts 34 have angles of curvature that decrease sequentially. The kit may include the holding bracket 102 for assessing the angulation of the patient's erect penis.

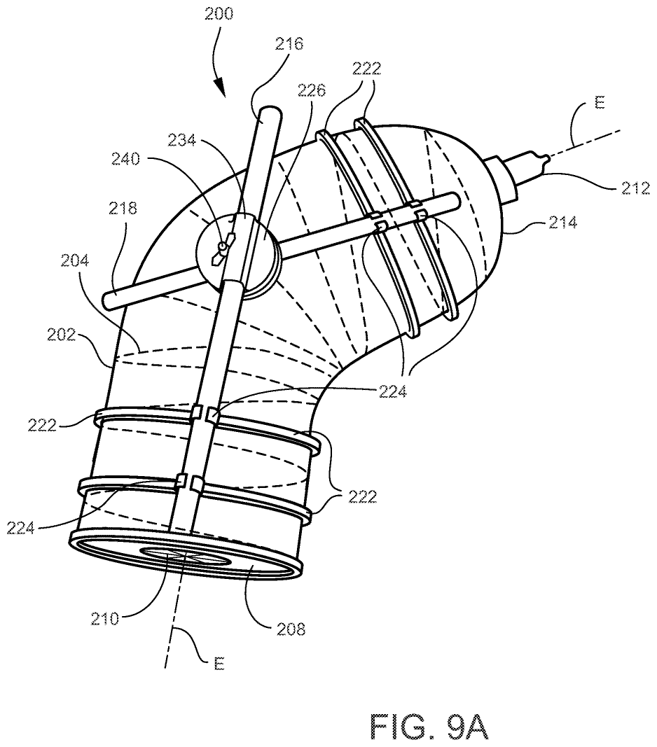

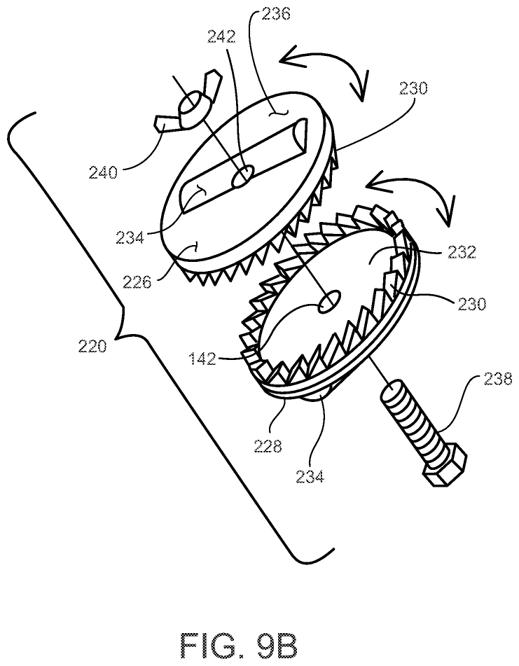

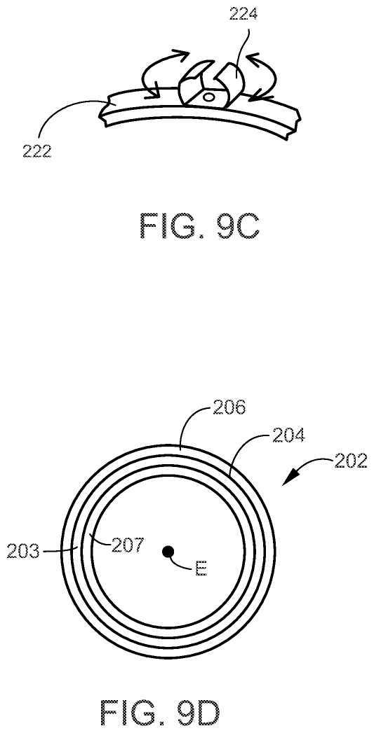

FIG. 9A illustrates another embodiment of an apparatus for treating a patient with Peyronie's disease. The apparatus includes a pliable vessel 200 having a central longitudinal axis E. As best seen in FIG. 9D, the pliable vessel includes a tubular portion 202 made of a spiral coil 204 embedded in an interior layer 203 of a first material and an exterior layer 206 of a second material and of a strength to maintain a vacuum therein. The spiral coil 204, interior layer 203 and exterior layer 206 may be made of the same or different materials. In one embodiment, the spiral coil 204, interior layer 203 and exterior layer 206 are all made of PVC. An example of suitable materials for the tubular portion 202 is HYDROMAXX™ heavy-duty flexible PCV suction and discharge hose that is available in a variety of diameters. See, for example, https://www.homedepot.com/p/HYDROMAXX-3-in-Dia-x-50-At-Green-Heavy-Duty-Flexible-PVC-Suction-and-Discharge-Hose-6107300050/310911351#overlay (last accessed on Apr. 16, 2023). Exemplary internal diameters of tubular portion 202 are, for example, 2.5 and 3 inches, depending on the patient. Of course, other diameters may be used if needed. If the interior layer 203 is not soft enough for a particular application and/or patient, an inner lining 207 of a soft polymer or plastic may be used.

The tubular portion 202 tapers at the top end 214 to close the tubular portion. The interior of the tubular portion 202 is configured to encapsulate an entirety of an angulated erect penis of the patient from a base of the angulated erect penis to a tip of the angulated erect penis. A bottom closure 208 seals the bottom of the tubular portion 202. A diaphragm 210 is disposed in the bottom closure 208 for insertion of a penis of the patient into the interior of the tubular portion 202. Diaphragm 210 may be similar in construction to diaphragm 54 (FIGS. 5A and 5B). A vacuum pump connection 212 is connected to the interior of the tubular portion 202 and may include an internal fluid lock-out valve 49 and a release valve 52 (FIGS. 3 and 4). The vacuum pump connection 212 may preferably be connected to the interior of the tubular portion 202 at a point corresponding to the tip of the angulated erect penis.

The pliable vessel 200 may include one or more bending assemblies. The bending assembly changes the angle of the central longitudinal axis E of the pliable vessel 200 and thereby bends the pliable vessel such that an interior surface of the tubular portion 202 contacts and applies pressure to the angulated erect penis of the patient at least at an area of undesired angulation of the angulated erect penis to thereby gradually break down fibrotic plaque and gradually reshape the angulated erect penis. The bending assembly includes first and second rods 216, 218 that intersect at an angle adjustment assembly 220 (shown in exploded view in FIG. 9B). The rods 216, 218 may be made of, for example, hard plastic, aluminum, or another strong, light-weight material.

The bending assembly includes at least one rod-holding ring 222 for each of the first and second rods 216, 218. FIG. 9A shows two rod-holding rings 222 for each of the first and second rods 216, 218. Each rod-holding ring 222 encircles the tubular portion 202, is fixed or clamped to the tubular portion 202 and includes a swiveling snap 224 (FIG. 9C) for retaining the rods 216, 218 to their respective rod-holding rings 222. The rod-holding rings 222 may be made of, for example, hard plastic, aluminum, or another strong, light-weight material.

The angle adjustment assembly 220 includes a pair of disks 226, 228. Each of the disks 226, 228 includes teeth 230 circumferentially located on surfaces 232 of the disks that face each other. The teeth 230 on one disk engage the teeth on the other disk. A sleeve 234 is fixed on each of the non-facing surfaces 236 of the disks 226, 228. Each sleeve 234 receives one of the rods 216, 218 and allows translation of the rod through the sleeve. A fastener removably fixes the pair of disks 226, 228 together. The fastener may include a threaded bolt 238 and threaded nut 240. The bolt 238 may pass through openings 242 in disks 226, 228.

The location of the angle adjustment assembly 220 on the pliable vessel 200 may be changed by translating the angle adjustment assembly along one or both or rods 216, 218 via sleeves 234. The angle of the angle adjustment assembly 220 may be changed by loosening the nut 240 from the bolt 238; disengaging the teeth 230 of the two disks 226, 228; rotating one of the disks with respect to the other disk to obtain the desired angle between rods 216 and 218; re-engaging the teeth of the two disks, and re-attaching the nut 240 to the bolt 238. Additional bending assemblies may be used simultaneously on the pliable vessel 200 in cases where the angulated penis is angulated in more than one plane.

A manually-operated or powered vacuum pump is removably attached to the vacuum pump connection 212 for creating the vacuum in the tubular portion 202. When the angulated penis is inserted through the diaphragm 210 and into the tubular portion 202, the vacuum pump creates a vacuum causing erection of the angulated penis in the tubular portion. The interior of the tubular portion 202 contacts and applies pressure to the angulated erect penis of the patient at least at an area of undesired angulation of the angulated erect penis to thereby gradually break down fibrotic plaque and gradually reshape the angulated erect penis. After enough treatment, the degree of angulation of the erect penis outside of the tubular portion gradually becomes the same degree as the degree of angulation of the pliable vessel. At that point, if the penis requires further straightening, the angle adjustment assembly 220 may be adjusted to a reduced angle and the process repeated until the penis is considered clinically straight.

As an example, a proposed course of a treatment may include using the pliable vessel 200 for about thirty minutes per day every day. The angle of the angle adjustment assembly 220 may be decreased every 30 days or so, depending on the change in angulation. Total treatment time may be, for example, in the six month range, but it may be shorter or longer based on the initial degree of angulation and other patient factors.

In contrast to prior art apparatus and methods, it is believed that the gentle, gradual modeling of the erect penis in accordance with the invention may maximize benefit while limiting risks and/or discomfort to the patient. Embodiments of the invention have been described to explain the nature of the invention. Those skilled in the art may make changes in the details, materials, steps and arrangement of the described embodiments within the principle and scope of the invention, as expressed in the appended claims.

What is claimed is:

1. An apparatus for treating a patient having Peyronie's disease, comprising:
A housing that is hollow;
a first insert removably disposed m the housing, the first insert having a cavity for insertion of an angulated penis of a patient wherein a degree of angulation of the cavity is less than an initial degree of angulation of an angulated erect penis of the patient;
a first fluid conduit in the first insert, the first fluid conduit having one end that connects with the cavity wherein the first fluid conduit extends to an exterior of the first insert;
the housing including a first end that is closed to define a vacuum chamber in the housing between the first insert and the first end of the housing;
a second fluid conduit having one end that extends through the first end of the housing to the vacuum chamber and a second end that is external to the housing:
a release valve located in the second fluid conduit external to the housing for selectively releasing a vacuum in the vacuum chamber; and
a diaphragm having an opening therein for insertion of the angulated penis in the cavity, the diaphragm being located at a second end of the housing corresponding to a base of the angulated erect penis to thereby provide a vacuum seal for the cavity.

2. The apparatus of claim 1, wherein the cavity completely encapsulates the angulated erect penis from the base of the angulated erect penis to a tip of the angulated erect penis and the cavity has a shape such that an interior of the cavity contacts and applies pressure to the angulated erect penis at least at an area of undesired angulation to thereby gradually break down fibrotic plaque and gradually reshape the angulated erect penis.

3. The apparatus of claim 1, wherein the housing and the first insert are cylindrical.

4. The apparatus of claim 1, wherein the one end of the first fluid conduit connects with the cavity at a point corresponding to a tip of the angulated erect penis.

5. The apparatus of claim 2, further comprising a vacuum pump removably attached to the second end of the second fluid conduit for creating the vacuum in the vacuum chamber and the cavity:
wherein, when the angulated penis is inserted through the diaphragm and into the cavity, the vacuum pump creates a vacuum causing erection of the angulated penis in the cavity, a gradual break down of the fibrotic plaque in the angulated erect penis and a gradual reshaping of the angulated erect penis so that a degree of angulation of the angulated erect penis outside of the cavity gradually becomes a same degree as the degree of angulation of the cavity.

6. The apparatus of claim 1, further comprising a plurality of removable inserts for sequential disposal in the housing, each of the plurality of removable inserts including a cavity for receiving the angulated penis wherein degrees of angulation of respective cavities of the plurality of removable inserts are a sequence of decreasing degrees of angulation to thereby gradually straighten the angulated erect penis of the patient and further wherein shapes of the respective cavities are such that interiors of the respective cavities contact and apply pressure to the angulated erect penis at least at an area of undesired angulation.

7. The apparatus of claim 1, wherein the degree of angulation in the cavity is in a range of about 5% to about 15% less than the initial degree of angulation of the angulated erect penis of the patient.

8. The apparatus of claim 6, wherein a decrease of the degree of angulation between two sequential inserts in the plurality of removable inserts is in a range of about 5% to about 15%.

9. The apparatus of claim 1, further comprising a ledge extending around an inner circumference of the housing and a flange extending around an outer circumference of the first insert wherein the flange rests on the ledge.

10. The apparatus of claim 1, wherein the diaphragm is disposed in an opening in a bottom of the second end of the housing.

11. The apparatus of claim 1, further comprising a lid that closes the first end of the housing.

12. (canceled)

13. The apparatus of claim 6, wherein the first insert and the plurality of removable inserts are made of a rubberized silicon material.

14. A method of treating a patient having Peyronie's disease, the patient having an angulated erect penis with a first degree of angulation, the method comprising:
providing a plurality of inserts, each insert having a cavity formed therein, each cavity formed to receive and encapsulate an entirety of the angulated erect penis of the patient from base to tip wherein degrees of angulation of respective cavities of the plurality of inserts are a series of sequentially decreasing degrees of angulation that are all less than the first degree of angulation of the angulated erect penis to thereby gradually break down fibrotic plaque and straighten the angulated erect penis:
inserting the angulated penis of the patient into the cavity of a first one of the plurality of inserts having a largest degree of angulation that is smaller than the first degree of angulation of the angulated erect penis:
creating a vacuum in the cavity of the first one of the inserts to cause the angulated penis to become erect such that the cavity encapsulates the entirety of the angulated erect penis from the base to the tip, and an interior of the cavity contacts and applies pressure to the angulated erect penis at least at an area of undesired angulation of the angulated erect penis;
maintaining the vacuum in the cavity of the first one of the inserts with the angulated erect penis inserted therein for a period of time to gradually break down fibrotic plaque at the area of undesired angulation and decrease the first degree of angulation of the angulated erect penis:
releasing the vacuum in the cavity; and
removing the angulated penis from the cavity.

15. The method of claim 14, further comprising placing the first one of the inserts in an external cylindrical housing that includes a vacuum chamber connected to the cavity of the first one of the inserts.

16. The method of claim 15, further comprising repeating the steps of inserting, creating a vacuum, maintaining the vacuum, releasing the vacuum and removing the angulated penis from the cavity until a degree of angulation of the angulated erect penis is a same degree as the degree of angulation of the cavity of the first one of the plurality of inserts.

17. (canceled)

18. The method of claim 14, further comprising,
inserting the angulated penis into the cavity of a second one of the plurality of inserts having a next largest degree of angulation that is smaller than the degree of angulation of the cavity of the first one of the plurality of inserts;
creating a vacuum in the cavity of the second one of the inserts to cause the angulated penis to become erect such that an interior of the cavity contacts and applies pressure to the angulated erect penis at least at the area of undesired angulation;
maintaining the vacuum in the cavity of the second one of the inserts with the angulated erect penis inserted therein for a period of time to gradually decrease the degree of angulation of the angulated erect penis:
releasing the vacuum in the cavity;
removing the angulated penis from the cavity; and
repeating the steps of inserting, creating a vacuum, maintaining the vacuum, releasing the vacuum, and removing the angulated penis from the cavity until a degree of angulation of the angulated erect penis is a same degree as the degree of angulation of the cavity of the second one of the plurality of inserts.

19. (canceled)

20. (canceled)

21. (canceled)

22. An apparatus for treating a patient having Peyronie's disease, the apparatus including a pliable vessel having a central longitudinal axis, the pliable vessel comprising:
a tubular portion made of a spiral coil embedded in an interior layer of a first material and an exterior layer of a second material and capable of maintaining a vacuum therein, the tubular portion tapering at a top end to close the tubular portion, an interior of the tubular portion configured to encapsulate an entirety of an angulated erect penis of the patient from a base of the angulated erect penis to a tip of the angulated erect penis;
a bottom closure that seals a bottom of the tubular portion;
a diaphragm disposed in the bottom closure for insertion of a penis of the patient into the interior of the tubular portion;
a vacuum pump connection connected to the interior of the tubular portion;
at least one bending assembly for changing an angle of the central longitudinal axis of the pliable vessel and thereby bending the pliable vessel such that an interior surface of the tubular portion contacts and applies pressure to the angulated erect penis of the patient at least at an area of undesired angulation to thereby gradually break down fibrotic plaque and gradually reshape the angulated erect penis.

23. The apparatus of claim 22, wherein the at least one bending assembly includes first and second rods that intersect at an angle adjustment assembly.

24. (canceled)

25. (canceled)

26. The apparatus of claim 22, wherein the vacuum pump connection is connected to the interior of the tubular portion at a point corresponding to the tip of the angulated erect penis.

27. The apparatus of claim 22, further comprising a vacuum pump removably attached to the vacuum pump connection for creating the vacuum in the tubular portion;
wherein, when the angulated penis is inserted through the diaphragm and into the tubular portion, the vacuum pump creates a vacuum causing erection of the angulated penis in the tubular portion and a gradual break down of the fibrotic plaque in the angulated erect penis and a gradual reshaping of the angulated erect penis so that a degree of angulation of the angulated erect penis outside of the tubular portion gradually becomes a same degree as a degree of angulation of the pliable vessel.

28. (canceled)

29. (canceled)
